(19) United States
(12) Patent Application Publication
(10) Pub. No.: US 2024/0325778 A1
(43) Pub. Date: Oct. 3, 2024

(54) DISPOSABLE PHOTOTHERAPY WRAP

(71) Applicant: MENNEN MEDICAL LTD, Yavne (IL)

(72) Inventors: Dan HAREL, Raanana (IL); Ben MANOACH, Willow Grove, PA (US)

(73) Assignee: MENNEN MEDICAL LTD, Yavne (IL)

(21) Appl. No.: 18/738,091

(22) Filed: Jun. 10, 2024

Related U.S. Application Data
(63) Continuation-in-part of application No. PCT/IL2022/051326, filed on Dec. 14, 2022.
(60) Provisional application No. 63/289,161, filed on Dec. 14, 2021.

(51) Int. Cl. A61N 5/06

(52) U.S. Cl. CPC A61N 5/0621; A61N 2005/0626; A61N 2005/0632; A61N 2005/0652; A61N 2005/0663

(57) ABSTRACT

A disposable phototherapy wrap comprising a plurality of light sources, wherein the disposable phototherapy wrap is configured to prevent direct contact and to provide sufficient electrical and heat isolation between the light sources and a body of a patient.

### CROSS REFERENCE TO RELATED APPLICATIONS

This is a continuation-in-part (CIP) of International Patent Application No. PCT/IL2022/051326, which has an international filing date of Dec. 14, 2022, and which claims priority from U.S. Provisional Patent Application No. 63/289,161 filed Dec. 14, 2021, all of which are incorporated herein by reference in their entirety.

### FIELD OF THE INVENTION

The present invention is in the field of phototherapy devices. More specifically, the invention relates to a disposable phototherapy wrap primarily used for patients needing phototherapy treatment, such as those with jaundice.

### BACKGROUND

Jaundice is one of the most common conditions that affect newborn babies. It is estimated that 6 out of every 10 babies develop jaundice, including 8 out of 10 babies born prematurely before the 37th week of pregnancy.

Bilirubin is a yellow pigment that is typically formed in the liver during the breakdown of old red blood cells. Newborn children often cannot effectively break down bilirubin in the liver, which can lead to neonatal jaundice. Accumulation of excess bilirubin in the human body can cause damage to the central nervous system, prompting the need for treatment. Phototherapy, or ‘light therapy’, uses light to isomerize the bilirubin transforming it into compounds that the newborn can readily excrete. The absorption spectrum of bilirubin lies primarily in the blue end of the visible spectrum, wherein the highest absorption wavelength is typically between 400-500 nm.

Phototherapy is the most common treatment clinicians use to treat newborn jaundice by lowering the bilirubin levels in the baby's blood through a process called photo-oxidation.

Photo-oxidation adds oxygen to the bilirubin, enabling it to dissolve easily in water. This makes it easier for the baby's liver to break down and remove the bilirubin from their blood. An aim of phototherapy is to expose the baby's skin to as much light as possible to optimize the break down and removal of bilirubin.

There are two main types of phototherapy solutions available at present:
1. conventional phototherapy—where the baby is laid under a halogen or fluorescent lamp with their eyes covered.
2. fibre-optic phototherapy—where the baby lies on a blanket that incorporates fibre-optic cables; light travels through the fibre-optic cables and shines onto the baby's back or wrapped around the baby.

Both methods require substantial capital investment per unit, and there are limitations in providing this service outside of a hospital setting.

An object of the present invention is to provide a solution that will eliminate the need for a capital expenditure budget altogether and offer a disposable solution instead, with a competitive and affordable cost per patient. Not only will such a solution be cost-effective and more hygienic, but it will also enable hospitals to treat as many babies as needed simultaneously without the need to buy many consoles like they have to with the current solutions in the market.

An object of the present invention is to provide a disposable body wrap with a built-in light source, such as light-emitting diodes (LED lights).

It is another object of the present invention to provide a disposable version of a standard phototherapy blanket that matches the efficacy of those devices that are already available in the market.

Other objects and advantages of the invention will become apparent as the description proceeds.

### BRIEF SUMMARY OF THE INVENTION

A disposable phototherapy wrap is primarily used for patients in need of phototherapy, such as those with jaundice. The disposable nature of the phototherapy wrap enables a more accessible solution for phototherapy, facilitates its use outside of a hospital setting, and reduces the overall cost.

A disposable phototherapy wrap comprising a plurality of light sources, wherein the disposable phototherapy wrap is configured to prevent direct contact and to provide sufficient electrical and heat isolation between the light sources and a body of a patient.

In one aspect, the disposable phototherapy wrap is structurally supported to maintain a distance between the plurality of light sources and a person undergoing phototherapy treatment.

In one aspect, the wrap is provided in the form of a blanket.

In one aspect, the plurality of light sources comprises light-emitting diodes (LEDs).

In one aspect, the plurality of light sources comprises glow sticks.

In one aspect, the disposable phototherapy wrap is biodegradable.

In one aspect, the intensity of the light from the plurality of light sources can be controlled and varied across the wrap.

In one aspect, the disposable phototherapy wrap further comprises an additional piece that extends underneath the person undergoing phototherapy treatment.

In one aspect, the disposable phototherapy wrap is provided in the form of a sleeping bag configured to incorporate a plurality of light sources, wherein said disposable sleeping bag is structurally supported to maintain a distance between the plurality of light sources and a person undergoing phototherapy treatment.

In one aspect, the disposable phototherapy wrap is shaped in alignment with the predetermined body areas of a person.

In one aspect, the disposable phototherapy wrap is configured in a laminar arrangement configured to enable light transfer toward the patient and to electrically isolate the body of the patient while underneath.

### BRIEF DESCRIPTION OF THE DRAWINGS

The above and other characteristics and advantages of the invention will be better understood through the following illustrative and non-limitative detailed description of embodiments thereof, with reference to the appended drawings, wherein:

FIG. 1 schematically illustrates a newborn under a phototherapy blanket, according to an embodiment of the invention;

FIG. 2 schematically illustrates a phototherapy blanket with LED strips, according to an embodiment of the invention;

FIG. 3 schematically illustrates a non-uniform positioning and non-uniform intensity of LED lights in a phototherapy blanket, according to an embodiment of the invention;

FIG. 4—a schematic cross-section of an arched phototherapy blanket, according to an embodiment of the invention;

FIG. 5—a schematic cross-section of an arched phototherapy blanket with support rods, according to an embodiment of the invention;

FIG. 6—a schematic cross-section of an arched phototherapy blanket with an additional blanket underneath the newborn, according to an embodiment of the invention;

FIG. 7—a schematic cross-section of an enclosed arched phototherapy blanket with an inner plastic shell, according to an embodiment of the invention;

FIG. 8A—schematically illustrates a laminar phototherapy wrap shaped to align with predetermined body areas of a person, according to an embodiment of the invention;

FIG. 8B schematically illustrates the plurality layers of the laminar phototherapy wrap of FIG. 8A, according to an embodiment of the invention;

FIGS. 9A-9B schematically illustrate a phototherapy wrap provided with wireless or wired operation authorization means, according to an embodiment of the invention;

FIG. 10 shows the multi-layer flexible circuit, in a body-wrap shape, with a plurality of LEDs installed, according to an embodiment of the invention;

FIG. 11 shows transparent nonwoven wrapper, designed to cover the multi-layer flexible circuit of FIG. 10, according to an embodiment of the invention;

FIG. 12 shows the multi-layer flexible circuit of FIG. 10 covered by the wrapper of FIG. 11;

FIG. 13 shows a multi-layer flexible circuit with an array of LEDs, designed to spread evenly intensive irradiance across the body-wrap with a controller, according to an embodiment of the invention;

FIG. 14 schematically illustrates a newborn baby positioned on an open body-shape wrap of FIG. 13; and

FIG. 15 schematically illustrates the newborn baby of FIG. 14 wrapped by the body-shape wrap.

### DETAILED DESCRIPTION OF EMBODIMENTS OF THE INVENTION

The present invention relates to a disposable phototherapy wrap comprising a built-in light source, such as a string of LEDs, for irradiating a target body area of a person, or the entire person's body. The proposed phototherapy wrap is configured to prevent direct contact and provide sufficient electrical and heat isolation between the light source and the patient's body.

Although the phototherapy wrap of this invention is disposable, it is not limited to a single use per se, i.e., as long as hygienic regulatory requirements are followed, then the phototherapy wrap may be used more than once. For example, the same disposable wrap can be used for multiple sessions with the same newborn baby that requires phototherapy.

Furthermore, although the primary mode of the disposable phototherapy wrap relates to its use in phototherapy for newborns, an expert in the art will see the opportunity afforded by this invention to be applied to any disposable device that utilizes an in-built light source. The invention is, therefore, not specifically limited to its use in phototherapy in newborns.

Reference will now be made to several embodiments of the present invention, examples of which are illustrated in the accompanying figures. Wherever practicable, similar or identical reference numbers may be used in the figures and may indicate similar or identical functionality. The figures depict embodiments of the present invention for purposes of illustration only. One skilled in the art will readily recognize from the following description that alternative embodiments of the structures illustrated herein may be employed without departing from the principles of the invention described herein. Furthermore, although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the invention as a whole.

As used herein, the terms “comprising” and “including” or grammatical variants thereof are to be taken as specifying the inclusion of the stated features, integers, actions, or components without precluding the addition of one or more additional features, integers, actions, components or groups thereof. This term is broader than and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub-embodiments “consist essentially of” and/or “consist of” the recited feature.

Furthermore, in keeping within the scope of the invention, it will be understood by one skilled in the art that an embodiment should not be considered only to include those aspects that are specifically depicted in that particular embodiment or example. An expert will see the opportunity to incorporate or remove different aspects of a particular example or embodiment without detracting from the scope of the invention.

In one embodiment, the light source in the phototherapy wrap is a ‘glow stick’, otherwise known as a ‘stick light’. In this embodiment, the light source has a one-time use, and the light source consists of a translucent or transparent plastic tube containing isolated substances that, when combined, make light. The implementation of this embodiment requires no external power source, and the wavelength of the luminescent source can be such that it provides the appropriate spectrum to carry out phototherapy as required. In this embodiment, any form of light emission can be utilized for this application, even ones that require no power source. In this embodiment, glow sticks, or the like, can be selected such that their color spectrum, intensity, and operation time are optimized, according to the needs of the patient undergoing phototherapy.

In one embodiment, the disposable wrap can utilize any suitable power source, such as those requiring a direct current (D.C.) or an alternating current (A.C.) source and/or one that is embedded and/or integrated into the wrap itself or external to it. A built-in D.C power source can include, but is not limited to, a plurality of single-use AA/AAA battery sources. An expert will understand that these single-use D.C batteries can be exchanged for other similar energy sources, such as rechargeable D.C batteries. According to some embodiments of the invention, the power source can be either a disposable or non-disposable element.

In another embodiment, the disposable wrap has an in-built electronic setup to facilitate plugging the wrap into a regular AC mains source. For the purposes of clarification, the term “in-built” is used herein in a broad sense, and is not meant to limit the scope. An expert will understand that this term can include the scope of the terms ‘embedded’, ‘integrated’, ‘incorporated’, or other non-limiting terms to describe a device that comprises elements within it. In this sense, the light system (e.g., a string of LED lights) can be incorporated inside the wrap itself or on either surface of the wrap therein. As such, in another embodiment, a control system or monitoring device can be incorporated into the wrap to control and monitor any data related to the inputs and outputs of the wrap. Furthermore, the wrap can contain a cloud-based controller and/or a monitor that monitors any inputs and outputs from the wrap and can display these data on a computer. Such data control and/or monitoring implementation can occur via any network, WiFi, internet, Bluetooth connection, cloud-based applications, or any other digital data-sharing platform.

The proposed disposable wrap may utilize any number of combinations of light sources for delivering light in predetermined patterns (i.e., to a predetermined body area) at a predetermined wavelength/frequency and intensity by a single disposable phototherapy wrap. For example, the wrap may incorporate several LED strips, LED lights, and/or glow sticks.

In another embodiment, the disposable wrap is waterproof.

The positioning, distribution and/or intensity of the light source in the wrap may be predetermined to be uniform or non-uniformed, for instance, to focus the light on the specific body part to be irradiated. In one embodiment, a string of LED lights is placed uniformly throughout the wrap. However, an expert in the art will see the opportunity to position the LEDs in different positions to optimize the conditions for phototherapy. As such, in another embodiment, the LEDs can be positioned to match the position of a newborn's body, or there could be a concentrated number of LEDs positioned above the newborn's chest. Furthermore, the intensity of the light from the light source can be distributed unevenly according to the needs of the patient. For the purposes of example alone, the light intensity can be greater in one region of the wrap in comparison to other areas.

In another embodiment, the wrap is a soft material and/or fabric that is comfortable to human touch. As such, any soft and/or flexible material can be used for the wrap, such as, but not limited to: cotton, polyester, woven acrylic, fleece, velvet, paper and silk, or any combination of soft materials and/or fabrics.

According to an embodiment of the invention, the disposable phototherapy wrap is designed with sustainability and practicality in mind, utilizing materials that ensure disposability while maintaining effectiveness. For instance, the wrap can be constructed from biodegradable nonwoven fabrics such as polylactic acid (PLA) or bamboo fiber, which are environmentally friendly and decompose under natural conditions without leaving harmful residues. Additionally, the inclusion of compostable plastics for any necessary rigid components, like connectors or embedded structures, further supports environmental sustainability. The LED strips, critical for the therapeutic function, are designed to be low-cost and efficient, using minimal electronic components that are easily separable from the fabric components for recycling purposes. The adhesives used in the assembly of the wrap are water-soluble or biodegradable, ensuring that they do not impede the recyclability or compostability of the wrap. This thoughtful selection of materials and modular design allows the wrap to be used effectively while still adhering to the principles of disposability and minimal environmental impact.

In one embodiment, the disposable phototherapy wrap is a sleeping bag that contains a flexible shell, enabling the plurality of light sources to be incorporated therein to maintain a distance from the newborn baby.

In another embodiment, the light sources are connected to a flexible printed circuit board within the phototherapy wrap.

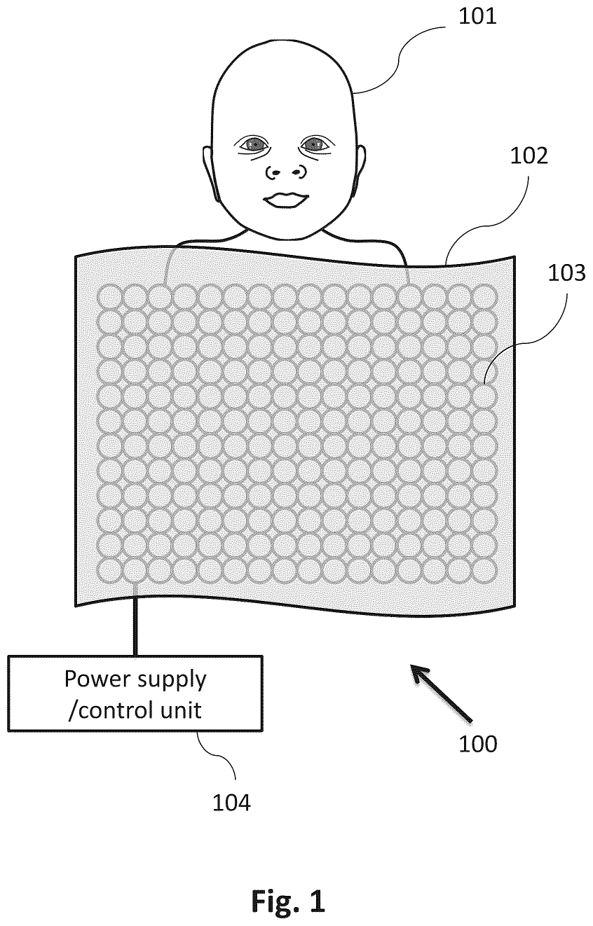

FIG. 1 shows the disposable phototherapy wrap configured as a phototherapy blanket 100, where a newborn baby 101 is positioned underneath the blanket. In the embodiment shown in FIG. 1, the disposable phototherapy blanket 100 comprises a fabric and/or material blanket 102 with a plurality of LED lights 103 incorporated therein. The plurality of LED lights 103 are all electronically connected to one another, typically in parallel, and are on the underside of blanket 102, i.e., facing the body of the newborn baby 101. A plurality of LED lights 103 are connected to a power supply and control unit 104, which can be incorporated externally or internally.

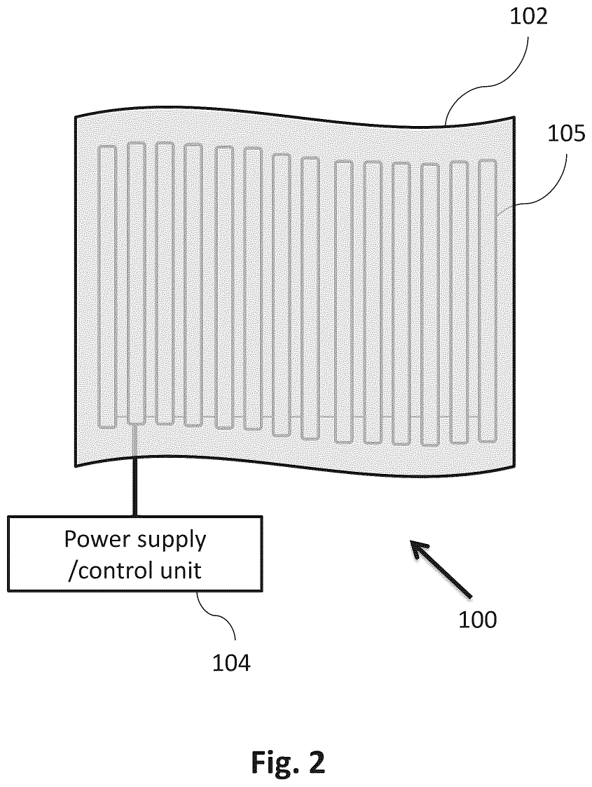

FIG. 2 shows the fabric and/or material blanket 102 as of FIG. 1 but wherein the plurality of LED lights at a different shape, e.g., strips 105. Again, these strips 105 are on the underside of blanket 102, configured to face towards the newborn baby, as in FIG. 1. The said embodiment can also incorporate a power supply and/or control unit 104.

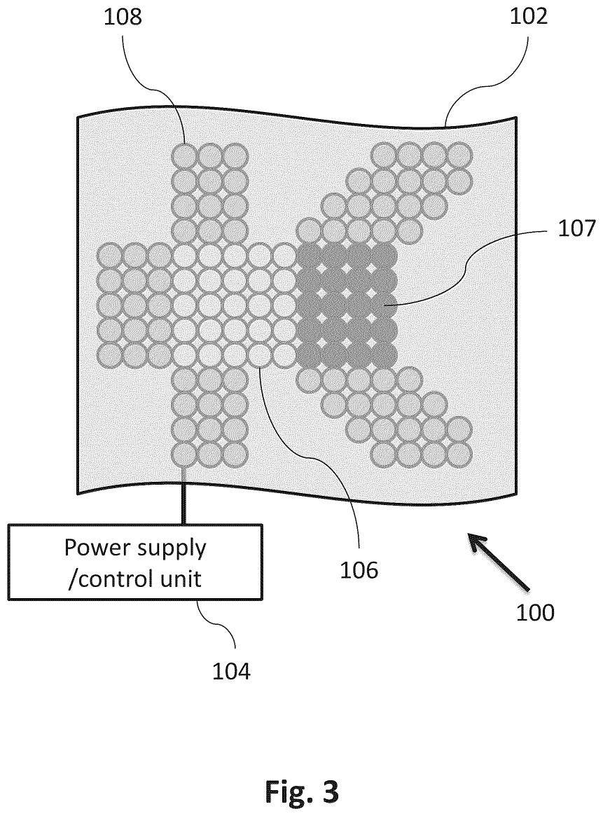

Arrays and strings of a plurality of uniform LED lights are commonly found in the marketplace and can be typically used in the present invention to save on costs and for ease of manufacturing. FIG. 3 shows an embodiment of the invention wherein a non-uniform positioning and intensity of a plurality of LED lights 106, 107, 108 are embedded within the disposable phototherapy blanket 102 of this invention. FIG. 3 depicts a patterning of a plurality of LED lights 106, 107, and 108 that coarsely outline the shape of a person. By using fewer lights and in a position that corresponds to the shape of a newborn baby, cost and energy can be saved in implementing the present invention. Furthermore, the power supply and control unit 104 can control the intensity of each individual LED in the plurality of LED lights 106, 107, 108. As such, different areas of the body can be specifically targeted for phototherapy. In one embodiment, the example outlined in FIG. 3 shows that one group of LED lights is turned on to full brightness 106, another group of LEDs is turned off 107, and another group of LEDs has medium brightness 108. Typically, a newborn baby will be wearing a diaper when undergoing phototherapy, and therefore, all the light directed toward the diaper region is wasted. Therefore, the embodiment shown in FIG. 3 is set such that no light is directed towards the newborn's diaper region, corresponding to a group of LED lights turned off 107 in FIG. 3, to save energy and costs. Furthermore, if the power supply is internal, rationing the use of light energy towards the newborn baby in this manner extends the lifetime of use of the disposable phototherapy blanket 100.

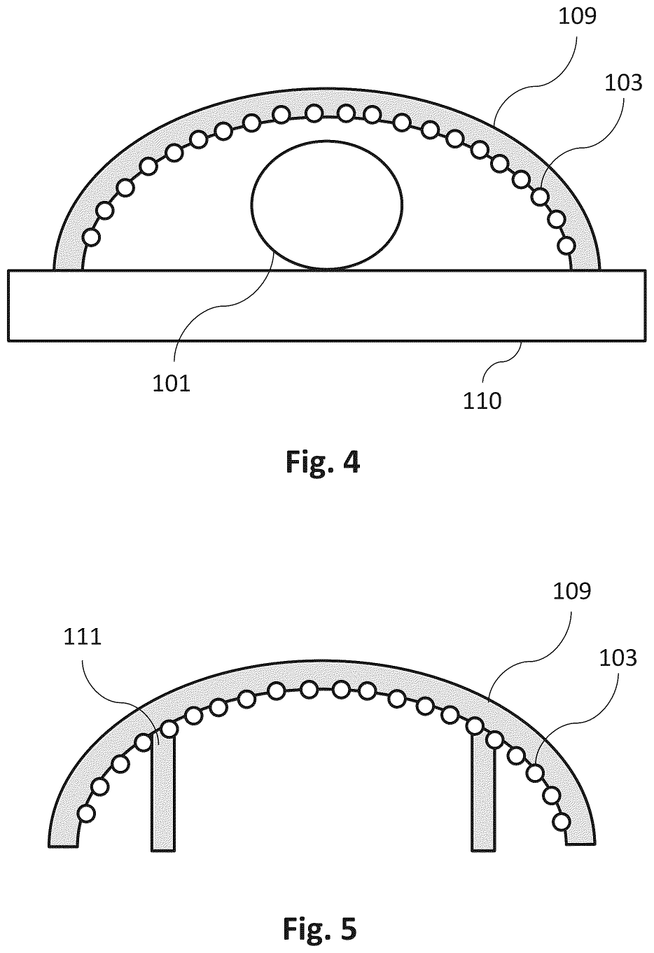

In one embodiment, depicted as a cross-section of the phototherapy blanket as illustrated in FIG. 4, the disposable phototherapy blanket is arched 109. In this embodiment, the newborn baby 101 is resting on a surface 110 (e.g., a bed, mattress, blanket, cushion, container, crib, cot, etc.), and the arched phototherapy blanket 109 is held at a distance from the newborn baby's 101 body. The plurality of LEDs 103 is shown to be on the underside of the arched blanket 109, facing the newborn baby 101. It will be understood by an expert in the art that the ‘arched’ shape detailed in this embodiment is for illustration purposes alone. Indeed, the phototherapy blanket can take on any shape that achieves the goal of the present invention, i.e., maintaining a distance between the light source that is incorporated into the blanket and the newborn baby itself.

One aspect of the present invention is to ensure that the newborn baby is kept safe during phototherapy. This means ensuring that the newborn baby doesn't overheat and is also not injured by contact with hot lights. In any case, LEDs do not heat up to the extent that incandescent lights do. Nonetheless, providing a distance between the phototherapy blanket 100 and the newborn baby 101 improves the safety specification of this invention. As such, a number of mechanisms can be used to hold the disposable phototherapy blanket at a distance from baby 101. A number of examples will now be presented but should in no way limit the scope of this aspect of the invention, namely, that there is a gap between the newborn baby and the phototherapy blanket. Any form of structural support, and mechanism thereof, to maintain a gap and/or distance between the plurality of LED lights and the person undergoing phototherapy is within the scope of this invention.

In one embodiment, the phototherapy blanket is configured to be a self-supporting arch 109. One method of ensuring an arched shape is to include further structural elements within the phototherapy blanket that act as a skeleton to ensure the phototherapy blanket maintains its distance from the newborn baby 101. In a further embodiment, a shape-memory alloy wire can be used as a frame around the phototherapy blanket 109 such that, when unfolded and/or released, the phototherapy blanket becomes arch-shaped. This mechanism is similar to a ‘pop-up tent’ wherein, when removed from the packaging or storage bag, the tent opens up and maintains its open structure.

In a further embodiment, plastic elements (e.g., sticks, strips, nets, etc.) can be incorporated into the blanket itself to ensure a rigidity of structure that supports the blanket away from the body of the newborn. An expert in the art will understand that there will be additional routine elements that can be incorporated into this support structure to ensure that the blanket maintains a distance from the body of the newborn baby without detracting from the intended scope of this invention.

FIG. 5 shows a schematic cross-section of an arched disposable phototherapy blanket 109 with a plurality of LEDs 103 and additional support rods 111. In this embodiment, the support rods 111 serve the purpose of holding the disposable phototherapy blanket away from the newborn baby. Furthermore, such support rods can be detachable or incorporated into the blanket itself. In one embodiment, the support rods 111 can contain a rigid support structure that is encased in the fabric of the arched blanket 109, for example, in a pocket that is incorporated into the arched blanket 109. Furthermore, the support rods 111 can support themselves, when positioned upright, against the weight of the arched phototherapy blanket 109, all whilst aiding in maintaining a distance between the blanket and the newborn baby. As such, the present invention does not limit the support mechanism to one mechanism or one component alone. Any number of support rods can be used, and in different positions, to best achieve a supported blanket structure.

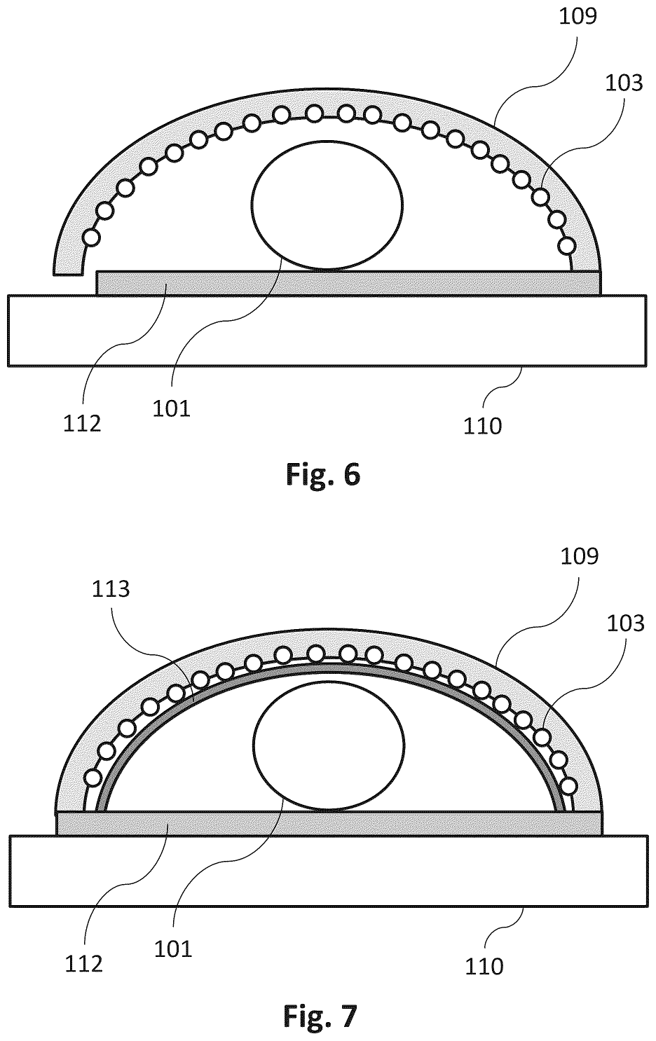

In one embodiment, the disposable phototherapy blanket only covers the newborn from above, as shown, for example, in FIG. 4. In another embodiment, shown in FIG. 6, the disposable phototherapy blanket covers the newborn baby from both sides, i.e., underneath it as well. In the embodiment of FIG. 6, a newborn baby 101 is resting on a support 110 (e.g., a table, mattress, blanket, crib, cot) and is enclosed by the disposable blanket 109 from above, with the incorporated plurality of LED lights 103, and is resting on an extension of the disposable blanket 112 which doesn't contain any LED lights. This embodiment provides an ‘all in one’ underside-and-overside-blanket-with-phototherapy capability. In a further aspect of this embodiment, the blanket underneath 112 can also incorporate an extruding blanket area, or integrated pillow, for the newborn's 101 head.

In another embodiment, shown in FIG. 7, a flexible shell 113 can be placed and/or attached inside the phototherapy blanket 109 to maintain a distance between the plurality of light sources 113 and the newborn baby 101. In this embodiment, the disposable phototherapy blanket can be made like a sleeping bag, wherein the baby is enclosed on all sides by the blanket but where the plastic shell 113 aids in maintaining a distance between the plurality of light sources 103 and the newborn baby 101. Furthermore, the underside of the sleeping bag 112 can be extended to support the newborn baby's 101 head and/or incorporate a pillow.

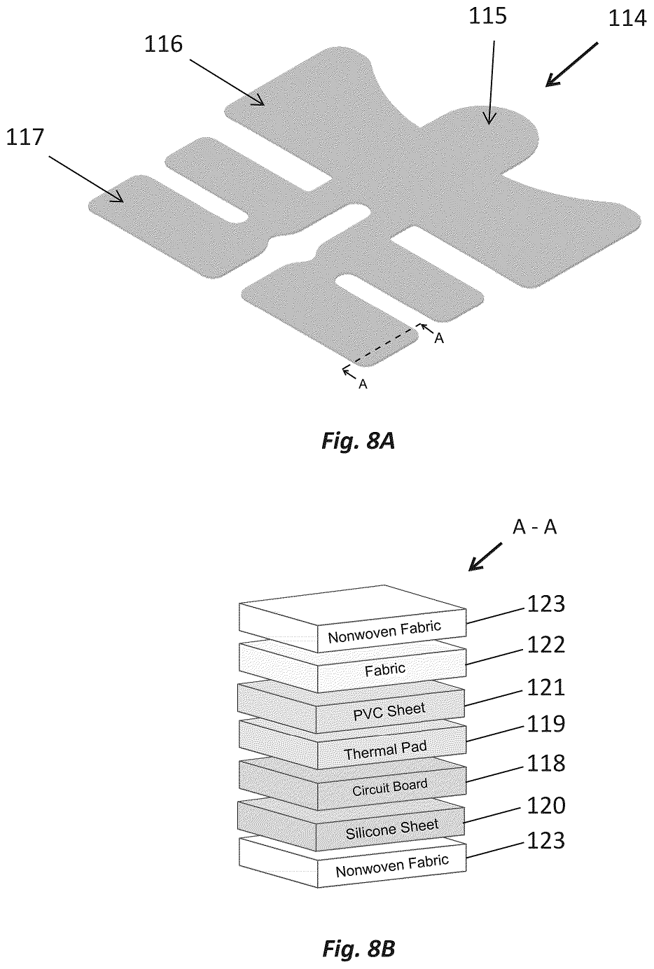

Now referring to FIGS. 8A-8B, which schematically illustrate an exemplary configuration of a disposable phototherapy wrap 114, according to an embodiment of the invention. In this embodiment, phototherapy wrap 114 is shaped in alignment with the predetermined body areas of a person. For instance, wrap section 115 is intended to cover a person's neck area, section 116 is intended to cover the person's chest and arms, and section 117 is aimed to cover/wrap the person's lower body and legs area.

Further illustrated in FIGS. 8A-8B is a section view A-A showing the laminar configuration of wrap 114, according to an embodiment of the present invention. The core of wrap 114 comprises a flexible circuit board layer 118 (e.g., comprising LEDs in a similar layout as LEDs 106-108 of FIG. 3 and their ancillary power supply and control circuitry) and an ancillary thermal pad layer 119, which disperses and thereby moderates the heat generated by circuit board 118.

A transparent silicone layer 120 is deployed underneath circuit board layer 118 for passing the therapeutic light toward the patient while electrically isolating between the patient and circuit board 118. A PVC laminate 121 is deployed above circuit board layer 118 to complete the electrical isolation thereof. A Fabric laminate 122 is deployed above PVC laminate 121 to block undesired light emission through the top of wrap 114.

Finally, nonwoven fabric layers 123 are utilized for providing comfortable padding and as hygiene means for preventing the spread of bacteria and infections. The abovementioned layers prevent direct contact and provide electricity and heat isolation between the heat-generating circuit board layer 118 and the person underneath, thus ensuring the person's safety and comfort.

Wrap 114 may be configured as a blanket, a pad, or any other laminar sheet aligned with a human body outline or exceeding it for further covering or completely wrapping a person's body.

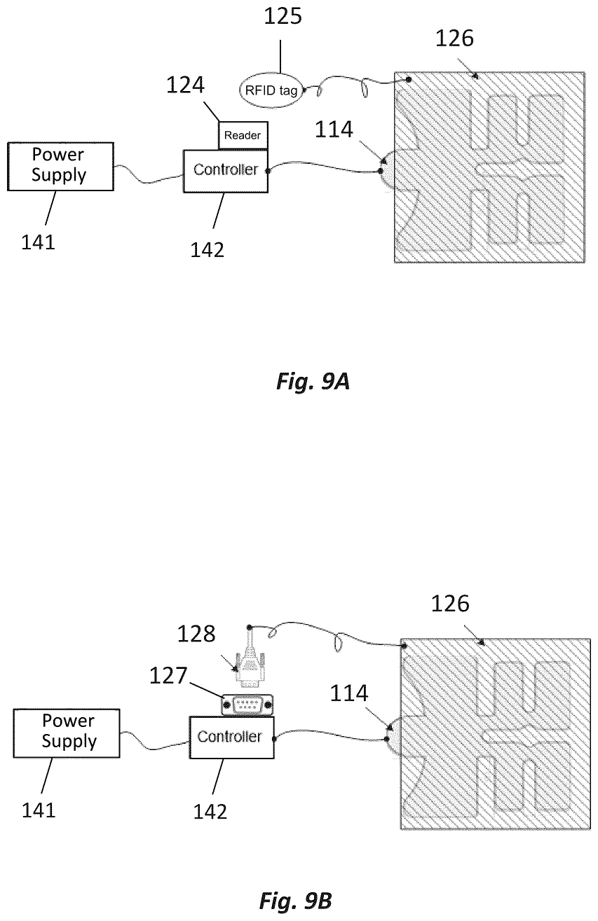

FIGS. 9A-9B illustrate exemplary wireless (FIG. 9A) and wired (FIG. 9B) operation authorization means of a disposable phototherapy wrap 114. According to one embodiment, a power supply 141 and a control unit 142 (also explained hereinabove with reference to power supply and control unit 104 of FIGS. 1-3) of wrap 114 comprises is provided with a wireless Radio Frequency Identification (RFID) reader which is configured to detect a predetermined RFID tag 125 and following the detection thereof, enables the operation of wrap 114. RFID tag 125 is physically attached to a storage casing 126 of wrap 114.

According to another embodiment, power supply and control unit 104 of wrap 114 is provided with an EPROM connector 127 and a suitable detection circuitry for enabling the operation of wrap 114 following attachment of an identification plug 128, for instance, where plug 128 comprises an electronic board which stores a predetermined identifier code set to be detected by the circuitry of connector 127.

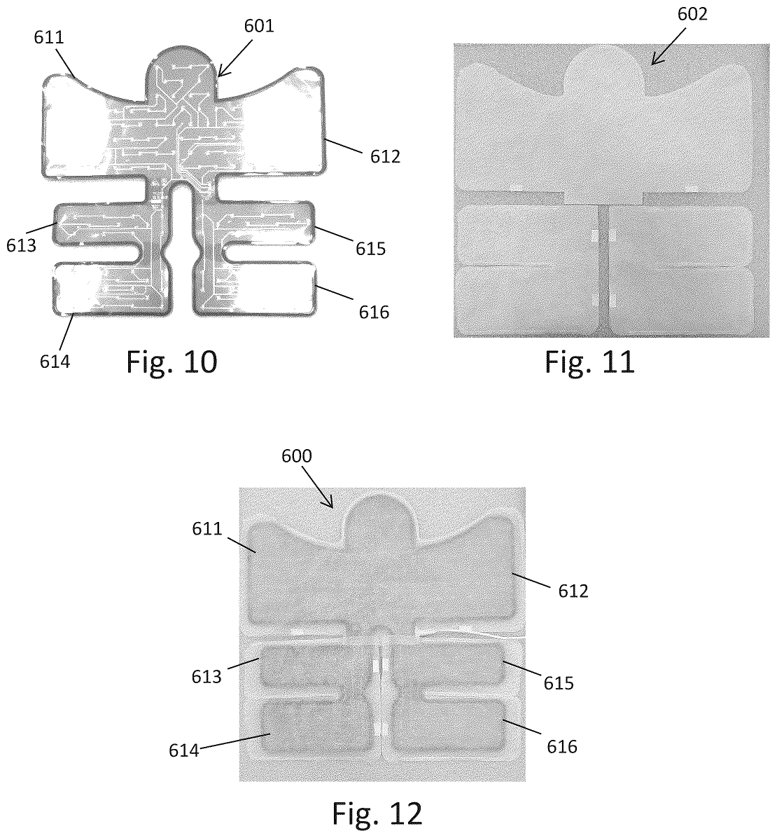

According to an embodiment of the invention, the disposable phototherapy wrap 600, as illustrated in FIGS. 10 through 15, is anatomically designed to align precisely with predetermined body areas of a person, specifically tailored for newborns. This innovative design is segmented into multiple sections 611-616, each meticulously adapted to wrap around different parts of the newborn's body, ensuring effective coverage while accommodating the natural movements of the baby.

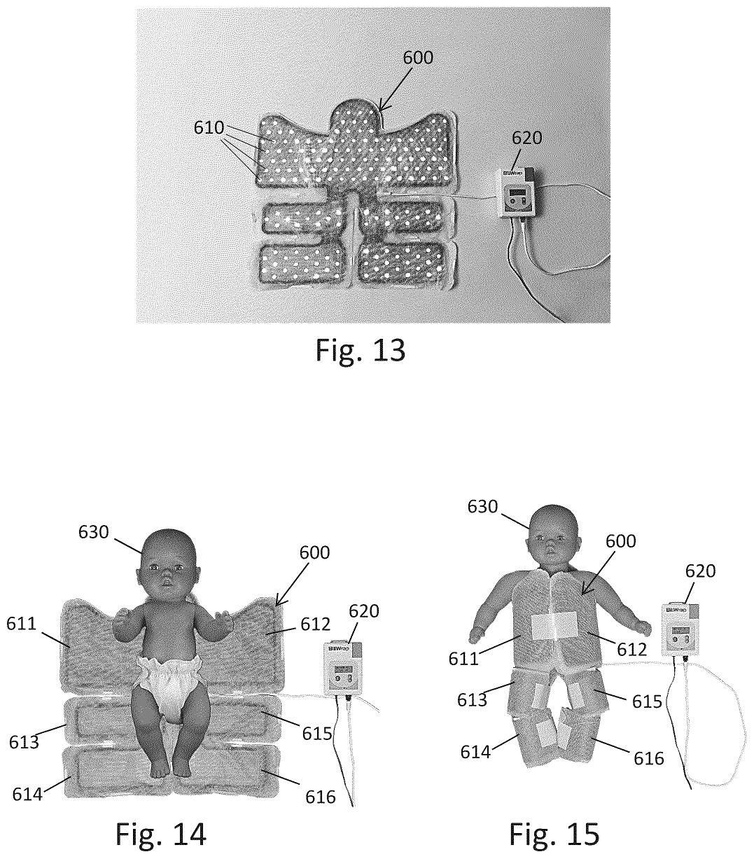

FIG. 10, showing a multi-layer flexible circuit 601 in a body-wrap shape, highlights the segmented design where each section of LEDs (e.g., as indicated by numeral 610 in FIG. 13) is strategically placed to align with key areas requiring therapeutic light exposure (as indicated by the multiple sections 611-616). The wrap's structure is not uniform but is instead divided into specific sections such as a torso section 611, 612, which wraps around the newborn while allowing space under the armpits. This design ensures the newborn's hands can move freely, reducing discomfort and potential distress.

In addition to the torso section, the wrap includes specialized sections for each leg, divided into upper and lower portions, as indicated by numerals 613, 614 and 615, 616. The upper left leg section 613 and the corresponding upper right leg section 615 are designed to wrap comfortably around the thighs, whereas the lower leg portions 614, 615 wrap around the calves. This careful division ensures that each part of the newborn's body receives optimal light exposure without compromising on comfort or safety. FIG. 11 shows a transparent nonwoven wrapper layer 602, designed to cover the multi-layer flexible circuit of FIG. 10, according to an embodiment of the invention, and FIG. 12 shows the multi-layer flexible circuit 601 of FIG. 10 covered by the wrapper 602 of FIG. 11.

FIGS. 14 and 15 further demonstrate the practical application of this anatomically aligned wrap 600. FIG. 14 shows the wrap 600 open, with the newborn 630 positioned in readiness for wrapping. This setup allows for an easy and secure wrapping process. Following this, FIG. 15 illustrates the newborn 630 fully wrapped, with each section 611-616 of wrap 600 conforming to different parts of the body as intended. The wrap's anatomical design not only enhances therapeutic effectiveness by ensuring that the light sources are optimally positioned but also maintains the newborn's comfort by accommodating natural body contours and movements.

This anatomically shaped design, represented across FIGS. 10 to 15, underscores the wrap's sophistication in combining therapeutic efficacy with ergonomic considerations, making it a novel solution in the field of newborn phototherapy.

In the embodiment illustrated in FIGS. 13 through 15, the phototherapy wrap includes a controller 620 designed to meticulously regulate the therapeutic operations of the wrap. This controller 620 is integral to ensuring that wrap 600 delivers light therapy in a precise and controlled manner, for achieving optimal therapeutic outcomes. The controller 620 is configured to adjust various parameters of the light emitted by the LEDs, such as intensity, duration, and even specific wavelengths, to cater to the particular medical needs of the newborn. For example, controller 620 can modulate the light intensity to prevent overexposure, or adjust the light's wavelength to target specific conditions more effectively, such as the optimal blue light spectrum for treating neonatal jaundice. Additionally, controller 620 is equipped with sensors (not shown) that monitor the real-time effectiveness of the therapy, allowing for automatic adjustments to maintain the desired therapeutic effects continuously. According to an embodiment of the invention, the controller's 620 ability to communicate with external devices via wireless or wired connections also enables remote monitoring and adjustment, providing healthcare professionals with the capability to oversee and modify treatment protocols as necessary. This embodiment ensures that the phototherapy wrap 600 is not only effective but also adaptable to the dynamic needs of neonatal care, enhancing both safety and efficacy in treating conditions like jaundice in newborns.

According to an embodiment of the invention, the disposable phototherapy body wrap 600 comprises an in-built temperature probe (not shown) that continuously monitors the temperature of the circuit within the wrap 600. This feature is designed to enhance safety by preventing overheating, which is especially important in neonatal care settings. If the temperature of the circuit exceeds a predetermined safe threshold, controller 620 is equipped with an automatic shutdown mechanism that deactivates the lights, thereby mitigating any risk of burns or discomfort to the newborn. This proactive safety mechanism ensures that the phototherapy wrap 600 remains within the safe operating temperatures, providing peace of mind to caregivers and healthcare professionals alike.

According to an embodiment of the invention, the design of the disposable phototherapy body wrap 600 also focuses on minimizing the exposure of LED light to the surrounding environment. This is achieved through the strategic placement of LEDs 610 and the use of directional light-emitting techniques that focus the light precisely where it is needed—the affected areas of the newborn's body. Additionally, the wrap incorporates light-absorbing materials around the edges and back of the LEDs to prevent light spillage. This design consideration ensures that the therapeutic light is concentrated on the patient, enhancing the efficiency of the treatment while protecting others in the vicinity from unnecessary light exposure.

According to an embodiment of the invention, directional light-emitting techniques are integral to the design of the disposable phototherapy body wrap, ensuring that therapeutic light is precisely focused where it is most needed while minimizing dispersion into the surrounding environment. For example, one effective technique involves the use of collimators, which are optical lens systems or reflective surfaces that narrow the beam of light emitted from the LEDs, directing it straight towards the treatment area. Another approach is the integration of light guides or fiber optics within the wrap, which channel light from the LEDs directly to specific points on the body, reducing spillage and enhancing the intensity of light on targeted areas. Additionally, the use of Fresnel lenses or prismatic film over the LED arrays can control the direction and spread of light, focusing it more accurately and efficiently. These methods ensure that the light emitted by the wrap is maximally utilized for therapeutic purposes, improving the efficacy of the treatment while safeguarding the comfort and safety of the patient.

According to an embodiment of the invention, to further ensure the safety and comfort of the newborn during phototherapy, the disposable phototherapy body wrap 600 is designed to effectively block the LED light from escaping the confines of the wrap. This is particularly important for protecting the delicate eyes of newborns during treatment. The wrap's material and design are such that it blocks out the light, making it unnecessary for the newborn to wear a protective mask during the therapy session. This feature not only provides safety against potential eye damage but also enhances the comfort of the baby, allowing them to receive treatment without the additional discomfort of wearing eye protection. To ensure effective confinement of LED light within the disposable phototherapy body wrap 600, several strategic design and material choices can be employed. For example, opaque materials can be integrated into the wrap's outer layers to prevent light from leaking, ensuring that only the designated treatment areas receive illumination. Reflective inner linings, made from materials like aluminum foil or reflective mylar, are utilized to redirect stray light back towards the patient's body, maximizing therapeutic efficacy. The edges of the wrap can be meticulously sealed, using techniques such as heat sealing or adhesive bonding with light-blocking properties, to eliminate any gaps through which light might escape. Additionally, light-absorbing materials can be incorporated around the perimeter of the LED layers to absorb any incidental light, using substances like carbon black infused fabrics or silicone with light-absorbing additives. The LEDs themselves are strategically placed to face inward, reducing outward light dispersion and enhancing focus on the treatment area. Moreover, the wrap features multiple fabric layers with the outermost possessing superior light-blocking capabilities, enhancing both functionality and patient comfort. Finally, a tight-fitting design ensures the wrap conforms closely to the body, minimizing spaces where light might escape, thereby allowing the newborn to undergo phototherapy without the need for wearing a mask, ensuring both safety and comfort.

These features collectively ensure that the phototherapy wrap is not only effective in treating conditions like jaundice but also safe and comfortable for the newborn, adhering to the highest standards of neonatal care.

All the above descriptions and examples have been given for the purpose of illustration and are not intended to limit the invention in any way. Many different mechanisms, methods of control, electronic and logical elements can be employed. all without exceeding the scope of the invention.

We claim:

1. A disposable phototherapy body wrap comprising a plurality of light sources mounted on a flexible substrate material, wherein each light source is configured to emit light within a therapeutic wavelength range optimized for the treatment of jaundice, wherein the phototherapy body wrap is anatomically shaped to align with predetermined body areas of a person, thereby covering a large surface area of the body.

2. The disposable phototherapy body wrap according to claim 1, wherein said body wrap is structurally layered to maintain a distance between the plurality of light sources and a person undergoing phototherapy treatment.

3. The disposable phototherapy body wrap of claim 1, wherein the plurality of light sources comprise light-emitting diodes (LEDs).

4. The disposable phototherapy body wrap of claim 1, wherein the intensity of the light from the plurality of light sources can be controlled and varied across the said wrap.

5. The disposable phototherapy body wrap of claim 1, further comprising an additional piece that extends underneath the person undergoing phototherapy treatment.

6. The disposable phototherapy body wrap according to claim 1, wherein the body wrap is shaped in alignment with predetermined body areas of the person comprising several sections, each intended to cover/wrap a different body area.

7. The disposable phototherapy body wrap according to claim 1, wherein the body wrap is configured in a laminar arrangement configured to enable light transfer towards the patient and to isolate the body of the patient underneath.

8. The disposable phototherapy body wrap according to claim 7, wherein the laminar arrangement comprises:
a) a flexible circuit board layer;
b) an ancillary thermal pad layer, which disperses and thereby moderates the heat generated by the flexible circuit board layer;
c) a transparent silicone layer deployed on the flexible circuit board layer for passing the therapeutic light toward the patient's body while electrically and thermally isolating between the patient and the flexible circuit board layer;
d) a PVC laminate deployed above the flexible circuit board layer to complete the electrical isolation thereof;
e) a fabric laminate deployed above the PVC laminate to block undesired light emission; and
f) a nonwoven fabric layer utilized for providing comfortable padding and as hygiene means for preventing the spread of bacteria and infections.

9. The disposable phototherapy body wrap according to claim 1, wherein the laminar arrangement prevents direct contact and provide electricity and heat isolation between the flexible circuit board layer and the body of the patient, thus ensuring the patient's safety and comfort.

10. The disposable phototherapy body wrap of claim 1, further comprising:
a built-in temperature probe configured to monitor the temperature of the wrap during use; and
an automatic shutdown mechanism operatively connected to the temperature probe, wherein the mechanism is configured to deactivate the light sources when the temperature exceeds a predetermined threshold, thereby ensuring safety by preventing overheating.

11. The disposable phototherapy body wrap according to claim 1, wherein the wrap is designed to minimize exposure of the surrounding areas to the LED light.

12. The disposable phototherapy body wrap according to claim 1, wherein the wrap is con figured to block emitted light from escaping the confines of the wrap, allowing for mask-free treatment of a patient.
